(19) United States
(12) Patent Application Publication
(10) Pub. No.: US 2026/0240501 A1
(43) Pub. Date: Aug. 20, 2026

(54) METHOD FOR DETERMINING A RISK OF CARDIAC DECOMPENSATION IN A PATIENT

(71) Applicant: CARDIAMETRICS, Caen (FR)

(72) Inventors: Cindy MICHEL, La Tronche (FR); Viateur TUYISENGE, Grenoble (FR)

(73) Assignee: CARDIAMETRICS, Caen (FR)

(21) Appl. No.: 19/146,287

(22) PCT Filed: Jan. 12, 2024
(86) PCT No.: PCT/FR2024/050045

§ 371 Date: Jul. 8, 2025

(30) Foreign Application Priority Data
Jan. 18, 2023 (FR) FR2300451

(51) Int. Cl. A61B 5/00; A61B 5/11; A61B 5/346

(52) U.S. Cl. CPC A61B 5/7246; A61B 5/1102; A61B 5/346; A61B 5/7203; A61B 5/7264; A61B 2562/0219

(57) ABSTRACT

A determination method for determining a risk of cardiac decompensation in a patient, including a step of processing an accelerometric signal acquired from the patient is, and determining a risk of cardiac decompensation based on the study of a plurality of markers, including segments of a processed accelerometric signal resulting from the processing. The step of processing the accelerometric signal comprises splitting the accelerometric signal into time cycles of the same duration; identifying, in each time cycle, segments characteristic of the cardiac sound, selecting the coherence of the segments identified, and calculating average segments.

The present invention relates to the field of medical devices and systems for monitoring the cardiac health of a living being. The present invention relates more particularly to a system for determining a decompensation of a cardiac failure within which an implantable medical device communicating with a computer server is configured to measure cardiac parameters. The cardiac failure is a chronic condition affecting a large proportion of the population, particularly the over-60s, and this affection may lead to episodes of decompensation, the frequency of which increases as the heart function deteriorates.

The cardiac failure decompensation episodes are often accompanied by emergency hospitalization of the patient suffering from this heart condition because they are not detected early enough, with the decompensation episode of the cardiac failure often appearing asymptomatic at its onset. This absence of symptoms detectable by the patient means that the decompensation episode of the cardiac failure progresses without any preventive action being performed. However, as soon as the patient begins to feel the first symptoms of the decompensation of the cardiac failure, such as fatigue, palpitations or shortness of breath, hospitalization is difficult to avoid.

It is clear from the above that the earlier an episode of cardiac failure decompensation is detected, the lower the risk of hospitalization, and the costs that this may generate, since early detection of cardiac failure decompensation in a patient means that the patient may be traited by prescribing medication allowing to stabilize the heart function of the patient.

The early detection of a cardiac failure decompensation may be based on in-depth analysis of various sub-clinical cardiac parameters, particularly hemodynamic parameters.

The present invention is part of this context and is intended to provide a system for determining an episode of cardiac failure decompensation in a patient.

The main object of the present invention is therefore a method for determining a risk of cardiac decompensation in a patient, during which a step of processing an accelerometric signal acquired from the patient is carried out and a step of determining a risk of cardiac decompensation based on the study of a plurality of markers, including segments of a processed accelerometric signal resulting from said processing step, is carried out the method being characterized in that the step of processing the accelerometric signal comprises:
a step of splitting the accelerometric signal into time cycles of the same duration,
a step of identifying, in each time cycle, segments characteristic of the cardiac sound, with a first search for a first type of segment in a first defined part of the time cycle and a second search for at least a second type of segment and a third type of segment in a second defined part of the time cycle,
a step of selecting the coherence of the segments identified, at least for the first types of segments and the second types of segments,
a step of calculating an average segment of a first type, an average segment of a second type and an average segment of a third type, the average calculation being based on all the segments of a first type selected, respectively all the segments of a second type selected, respectively all the segments of a third type selected,
   the method also being characterized in that the determination step comprises at least one step of determining an increase in the amplitude of the first type of segment relative to data in memory and/or a decrease in the amplitude of the third type of segment relative to data in memory.

The method according to the invention is thus particular in that it allows to determine a risk of decompensation on the basis of a cardiac sound representative of a given acquisition period, this representative cardiac sound resulting from a compilation of cardiac sounds obtained by processing at least one accelerometric signal.

The accelerometric signal is split into a plurality of time cycles of equal duration. By the same duration, it should be understood that the duration of the time cycles of an acquired accelerometric signal may be different from the duration of the time cycles of another accelerometric signal previously acquired on the same patient, but that the duration of the time cycles of the same accelerometric signal is the same for each time cycle at the end of the splitting step.

The duration may be defined before the method is implemented or it may be defined during the method, by calculation, as a function of the characteristics of the time cycles specific to the accelerometric signal being analyzed. In a non-limiting example of the invention, the accelerometric signal is first chopped into time cycles, using one identification parameter to start the splitting of the period and another identification parameter, or the same one, to finish the splitting of the period, so that each cycle has its own duration, reflecting the actual appearance of these identification parameters. The durations of the time cycles are then averaged to determine the duration to be given to the time cycles for the rest of the method according to the invention. And we adjust the duration of each time cycle resulting from the initial chop to give it a duration equal to this average duration. Depending on the initial duration of the time cycle, the control unit may either cut the time cycle to reduce its duration and give it a duration equal to the average duration, or lengthen the time cycle to increase its duration. In the latter case, the control unit is configured to add one or more zero signal values at the end of the time cycle, the values being added at regular intervals as a function of the sampling frequency. For example, at a sampling rate of 500 Hz, a zero signal value is added every two milliseconds at the end of the time cycle.

For each time cycle defined in the given acquisition period, different segments of the cardiac sound are identified and the accelerometric signal is advantageously processed by type of segment, with in particular the segments of the first type on one side, and the segments of the second type and of the third type on the other side, these last two types of segment being processed together or in parallel. This has the advantage of being able to retrieve a valid information on a segment of one type for a given time cycle, whereas the information relating to the segment of another type is to be excluded for the same time cycle, which allows to increase the number of segments of the same type to be compiled for an acquisition period. In other words, for a given time cycle, if no segment of the first type is identified or if the segment of the first type identified seems to be too different from what is observed in the other time cycles, in particular due to a one-off acquisition problem, it is possible to recover an information that may be used for the segment of the second type and/or for the segment of the third type.

According to an optional characteristic of the invention, the identification step comprises a sub-step of excluding segments previously identified as being segments of the first, second or third type, and classified during this exclusion sub-step as being artefacts.

In particular, such artefacts may occur when the signal is acquired simultaneously with a movement of the patient during sleep, and/or simultaneously with snoring or coughing, although these examples are not limiting of the invention. It is understood that segments identified as artefacts, i.e. segments which are too far from the shape of a physiological segment, either in duration or in amplitude, are excluded from the coherence selection step, which avoids the need for a control unit to spend computing time on segments which will not be retained after this selection step anyway and which may alter the analysis. This improves the speed of information processing and prevents the coherence selection step from being polluted by segments that are not characteristic of the condition of the patient.

According to an optional characteristic of the invention, the exclusion sub-step comprises an operation of calculating a spectral analysis score, the segments presenting a score greater than a threshold value being identified as being artefacts and excluded. In particular, the spectral analysis score is a z-score, and the threshold value is equal to 3.

According to an optional characteristic of the invention, the segments of the third type are detected in the second part of the time cycle following the detected segments of the second type.

According to an optional characteristic of the invention, the third type segments have a smaller average amplitude than the average amplitude of the second type segments.

According to an optional characteristic of the invention, the determination method comprises a step of acquiring at least one accelerometric signal from the patient, prior to the processing step.

According to an optional characteristic of the invention, the acquisition step is carried out by a three-axis accelerometer, the processing step being carried out for each of the signals detected on one of the three axes or for an overall signal resulting from the combination of the signals acquired on each of the axes.

According to an optional characteristic of the invention, the step of acquiring at least one accelerometric signal is accompanied by a step of acquiring an electrocardiogram signal.

According to an optional characteristic of the invention, the signal splitting step comprises an operation of synchronizing the accelerometric signal on the basis of the electrocardiogram signal.

According to an optional characteristic of the invention, the identification step comprises a step of calculating the envelope of the signal, a segment being identified when the amplitude of the envelope is greater than a determined threshold in a defined part of the time cycle.

According to an optional characteristic of the invention, the signal envelope is calculated over the entire cycle.

According to an optional characteristic of the invention, the threshold determined is of the order of 10% of the maximum amplitude value.

According to an optional characteristic of the invention, the envelope calculation step comprises both a squared envelope calculation and an absolute value calculation.

According to an optional characteristic of the invention, the first defined part of the time cycle in the identification step is equal to the first third of the duration of said time cycle, the second defined part of the time cycle being equal to the last two thirds of said time cycle.

According to an optional characteristic of the invention, the selection step comprises at least two series of correlation calculations performed in parallel, including a first series of correlation calculations carried out on all the segments of the first type and a second series of correlation calculations carried out on all the segments of the second type.

According to an optional characteristic of the invention, each series of correlation calculations performed for a given type of segment comprises a first calculation of the cross-correlation between all the segments identified as being segments of this type, in order to define a reference segment for this type of segment, followed by steps of calculating the correlation of each of the segments identified as being segments of this type with respect to the reference segment.

According to an optional characteristic of the invention, the segments selected in each series of correlation calculations are the segments which have a correlation index with the reference segment which is greater than or equal to a predetermined correlation threshold. By way of example, the correlation threshold is at least 50%, and for example of the order of 60%. In other words, all the segments with a correlation index below the correlation threshold, 60% in this example, are set aside and not considered for the average segment calculation step.

According to an optional characteristic of the invention, the selection step comprises only two series of correlation calculations performed in parallel, including a first series of correlation calculations carried out on all the segments of the first type and a second series of correlation calculations carried out on all the segments of the second type.

In this variant, no cross-correlation calculation is performed for the segments of the third type, even though they have already been identified. In this case, sorting by coherence in relation to a reference segment is only performed via segments of the second type. The third type segment is sorted in the same way as the second type segment present in the same time cycle: if a second type segment is retained after the coherence sorting step, the third type segment of the same time cycle is retained, while if a second type segment is excluded after the coherence sorting step, the third type segment of the same time cycle is also excluded.

In other words, a signal processing is carried out which aims to consider the third type segment because it is one of the most specific for determining a cardiac failure, but this signal processing is specific in that the average third type segment is calculated on the basis of third type segments which have been excluded or retained as a function of the exclusion or the retention of the second type segments of the same time cycle, the identification of these second type segments being more certain.

An accelerometric signal is segmented into three segments and the first segments on the one hand and the second and third segments on the other are processed in two separate procedures, with the second and third segments being processed by a cross-correlation and a selection based on the second segments alone.

According to an optional characteristic of the invention, the selection step comprises three series of correlation calculations performed in parallel, including a first series of correlation calculations carried out on all the segments of the first type S1, a second series of correlation calculations carried out on all the segments of the second type S2, and a third series of correlation calculations carried out on all the segments of the third type. In other words, cross-correlation calculations are performed for each of the segments identified beforehand.

According to an optional characteristic of the invention, between the selection step and the step of calculating the average segments, a step is performed for each type of segment, wherein the selected segments are reset in time to the corresponding reference segment.

According to an optional characteristic of the invention, the determination step further comprises a step of determining an increase in the amplitude of the second type of segment with respect to data in memory. Such an increase may be a sign of pulmonary arterial hypertension.

Other characteristics, details and advantages of the invention will become clearer on reading the following description, on the one hand, and examples of embodiments given by way of indication and non-limitation with reference to the attached schematic drawings, on the other hand, on which:

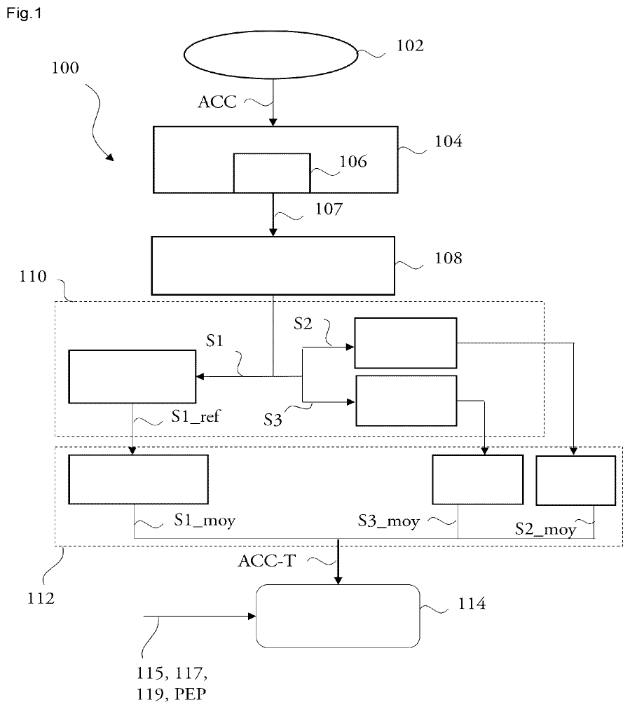

FIG. 1 shows the main steps of a method for determining a cardiac decompensation according to the invention;

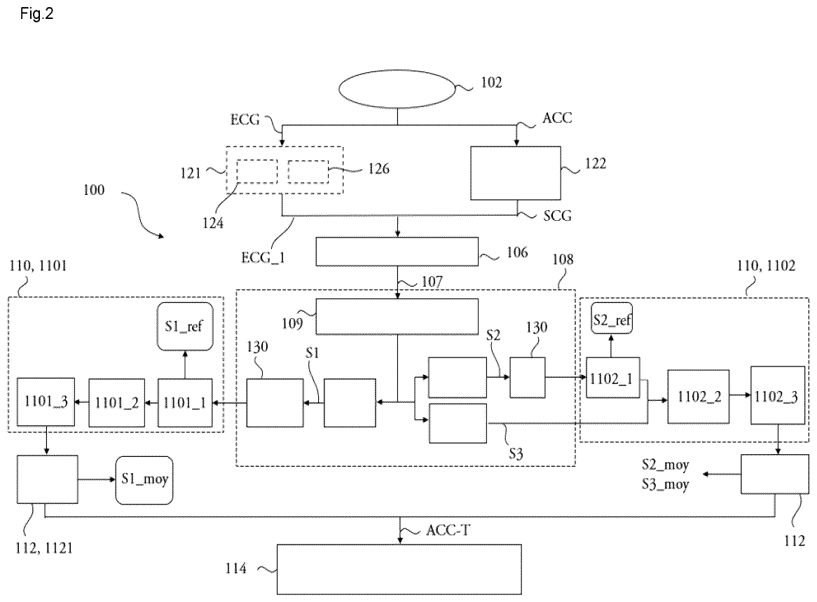

FIG. 2 shows a first embodiment of the method illustrated in FIG. 1;

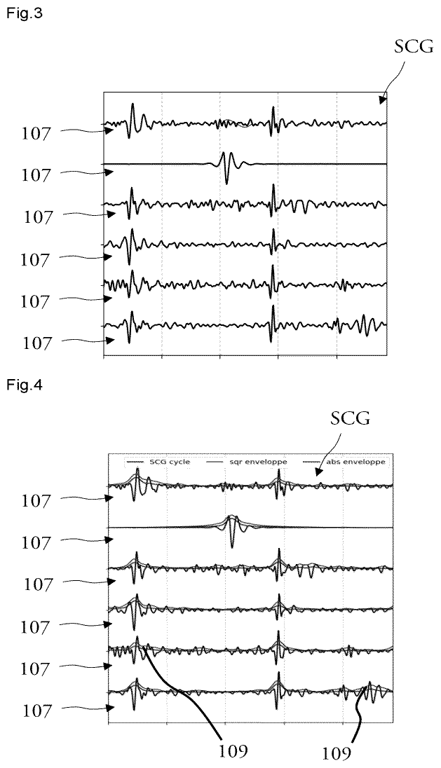

FIG. 3 shows an accelerometric signal split into time cycles during one of the steps in the method;

FIG. 4 shows the accelerometric signal from FIG. 3, with a squared envelope and absolute value envelope calculation allowing subsequent steps in the method to be implemented;

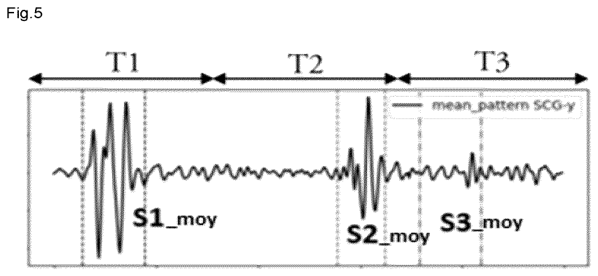

FIG. 5 shows an average time cycle representative of the processed accelerometric signal, with a segment of a first average type in a first third of the cycle and a segment of a second average type and a segment of a third average type in the other two thirds;

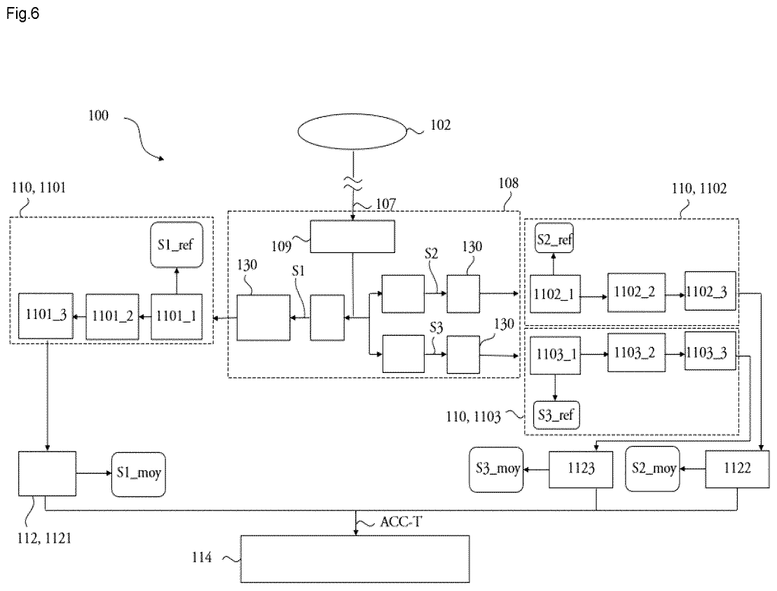

FIG. 6 represents a second embodiment of the method illustrated in FIG. 1.

First of all, it should be noted that although the figures set out the invention in detail for its implementation, these figures may of course be used to better define the invention, if necessary. It should also be noted that these figures only show examples of embodiments of the invention.

The characteristics, the variants and the different embodiments of the invention may be associated with one another in various combinations, insofar as they are not incompatible or mutually exclusive. In particular, it will be possible to imagine variants of the invention comprising only a selection of characteristics described hereinafter in isolation from the other characteristics described, if this selection of characteristics is sufficient to confer a technical advantage or to differentiate the invention from the prior art.

In the figures, the elements common to several figures retain the same reference.

FIG. 1 illustrates a method 100 for determining a risk of cardiac decompensation in a patient in accordance with the invention, in the course of which the main steps are a step of processing an accelerometric signal acquired from the patient and a step of determining a risk of cardiac decompensation based on the study of a plurality of markers including said processed accelerometric signal.

FIG. 1 illustrates more particularly the broad lines of the determination method 100 with a step 102 of acquiring at least one accelerometric signal ACC, a step 104 of processing the accelerometric signal which comprises at least a step 106 of splitting the accelerometric signal into time cycles 107 of a determined duration, a step of identifying 108, in each time cycle 107, segments S1, S2, S3 characteristic of the cardiac sound, a step of selecting 110 the coherence of the identified segments, a step of calculating 112 average segments representative of the processed accelerometric signal ACC-T, and the step of determining a risk of cardiac decompensation 114.

The step 102 of acquiring at least one accelerometric signal ACC may be carried out by any possible acquisition means, without this choice being restrictive of the present invention, as long as the chosen acquisition means allows the acquisition of a signal that is as representative as possible of cardiac activity, with as little artefact as possible. For example, to acquire such a signal, it may be advisable to provide a subcutaneous implant at the level of the ribcage and to plan the acquisition during the sleep of the patient, so as to ensure that the acquisition is carried out when the patient is not making any parasitic movements. Such physiological stability ensures the acquisition of repeatable data, so that the variations observed by the method according to the invention after processing the accelerometric signal may be considered to be due to the pathology.

The accelerometer used may be a three-axis accelerometer, which allows to acquire the same accelerometric signal representative of the cardiac activity along three separate axes for a given period. In the description that follows, the processing of an accelerometric signal ACC may be the processing of a signal recovered on a single axis, as a result of a selection from the three axes, or acquisition by an accelerometer capable of acquiring a signal along a single axis. However, it should be noted that the description would apply identically if the accelerometric signal processed were a signal resulting from a combination of the values acquired on each of the three axes.

The acquisition means used is configured to send the acquired data to a control unit, which may be installed on a remote server, on a mobile apparatus of the patient, or even on computing equipment connected to the acquisition means, without this being restrictive of the invention, the data may be transmitted by a wireless communication protocol, or by wired means if the type of acquisition means used and the location of the control unit allow this. The control unit is configured to implement the step 104 of processing the accelerometric signal ACC.

More particularly, the step of splitting 106 the accelerometric signal consists of evaluating the accelerometric signal ACC acquired over the whole of the acquisition period, which may be of the order of 30 seconds for example, and split it into time cycles 107 of the same duration. The time cycles have the same duration, in that this duration is the same for each time cycle resulting from the splitting of the same accelerometric signal, which is then analyzed in the rest of the method.

Means are described in the remainder of the description for generating time cycles of the same duration, and by way of example, the duration of the time cycles of the same accelerometric signal may be of the order of 300 to 1500 milliseconds.

As will be described below, in particular with reference to FIG. 2, this step of splitting 106 the accelerometric signal may advantageously be associated with the simultaneous processing of the accelerometric signal ACC and of an electrocardiogram signal ECG acquired simultaneously with said accelerometric signal, so that the particular characteristic of the cardiac activity, used to give at least one origin to the time cycles 107 of the accelerometric signal ACC and, where appropriate, the end of the time cycles, may be detected by analysis of the electrocardiogram signal ECG and may, for example, correspond to the appearance of a peak R.

Once the signal has been split into different time cycles 107, at least some of these time cycles are analyzed to identify, during the identification step 108, segments S1, S2, S3 characteristic of the cardiac sound. These characteristic segments are identified in particular when the amplitude of the accelerometric signal, and more particularly the amplitude of an envelope of the accelerometric signal obtained by a signal rectification operation, is greater than a defined value, for example greater than a percentage of a maximum value of amplitude of the envelope observed over the duration of the time cycle 107.

In particular, the identification step 108 allows to identify at least a first type of segment S1 in a first defined part of one of the time cycles 107 analyzed and at least a second type of segment S2 and a third type of segment S3 in a second defined part of this same time cycle 107. Thus, for a given acquisition period, the control unit is configured to store, on the one hand, all the segments of the first type S1 identified over all the time cycles 107 in an appropriate database, and to store in a distinctive manner in an appropriate database the segments S2, S3 identified in the second parts of each analyzed time cycle 107.

According to the embodiments described below, the segments of the second type S2 and the segments of the third type S3 may then be processed simultaneously, with the processing focusing solely on the segments of the second type S2, or they may be processed in parallel, with the processing focusing on both the segments of the second type S2 and the segments of the third type S3.

The next step is therefore carried out at least for the first types of segments S1 and for the second types of segments S2 and/or the third types of segments S3, with reference to what has been mentioned above, and consists of a step 110 of selecting the coherence of the segments identified. This step will be described in more detail below, with reference to FIGS. 2 and 3 in particular: in particular, it consists of cross-correlating all the segments of the same type identified beforehand in order to be able to deduce a reference segment for this type of segment and then to be able to keep only the segments which come closest to this reference segment, i.e. the coherent segments of each types of segment.

The processing of the signal ends with a step of calculating 112 an average segment for each of the segment types, regardless of the number of segment types that have been processed simultaneously in the previous step. In other words, whether the segments of the third type have been processed separately from the segments of the second type or have been attached to the latter, the calculation step allows to obtain an average segment of a first type S1_moy, an average segment of a second type S2_moy and an average segment of a third type S3_moy.

The processed accelerometric signal, resulting from the signal processing step and reflecting the cardiac activity of the patient over the signal acquisition period, in this case of the order of 30 seconds, is formed by juxtaposing each of the previously calculated mean segments S1_moy, S2_moy, S3_moy.

As mentioned, the processed accelerometric signal is a marker used in the step of determining a risk of cardiac decompensation 114. This determination step may in particular consider other markers, including electrophysiological markers 115 determined via the electrocardiogram signal mentioned above, the respiratory frequency 117, the inclination 119 of the body of the patient during the night or the pre-ejection period, referred by the acronym PEP.

The marker corresponding to the processed accelerometric signal may be used in particular to compare the amplitude of the average segment of the first type with data stored in memory relating to the evolution of segments of this first type over a given period and to compare the amplitude of the average segment of the third type of segment S3 with data stored in memory relating to the evolution of segments of this first type over a given period, which may be different from the period over which the segments of the first type are considered.

In particular, the determination step 114 may result in information about the risk of cardiac decompensation, when it is observed that the segment of first type S1 of the processed accelerometric signal ACC_T is less than what was previously recorded, and that the segment of third type S3 of the processed accelerometric signal ACC_T is greater than what was previously recorded.

The analysis of the heart sounds provides information on hemodynamic function. In the case of cardiac failure, the first cardiac sound, i.e. the first type S1 segment, and the third cardiac sound, i.e. the third type S3 segment, are both of interest for predicting decompensation.

The amplitude of the first type S1 segment is strongly correlated with ventricular contractility and overall cardiac performance. A reduction in amplitude indicates a reduction in cardiac contractility. Conversely, an increase in the amplitude of the third type S3 segment is a specific sign of an increase in filling pressures following a reduction in cardiac contractility. Several studies have shown that the specificity of an increase in the amplitude of the third type S3 segment in cardiac decompensation is very high, around 80%.

With reference to FIG. 2, a first embodiment of the invention will now be described.

As mentioned, the step 106 of splitting the accelerometric signal ACC into time cycles 107 of a given duration takes into account, in particular, in this embodiment, an electrocardiogram signal ECG. The result, prior to this splitting step 106, is a step for acquiring this electrocardiogram signal ECG, carried out simultaneously with the step of acquiring the accelerometric signal in a common acquisition step 102.

These two distinct signals are acquired synchronously, i.e. with simultaneous triggering of the acquisition operations, and with acquisition on the same time scale.

The control unit is configured to carried out a first signal analysis step 121 in parallel, during which the electrocardiogram signal ECG is analyzed, and a second signal analysis step 122, during which the accelerometric signal ACC is analyzed.

The first signal analysis step 121 comprises at least one step of filtering the electrocardiogram signal 124 to eliminate artefacts and a step of detecting signal peaks 126 representative of defined cardiac characteristics. The control unit is configured to define at the output of this first analysis step 121 at least one datum about the temporal position of these peaks relative to the start of the acquisition period.

The first signal analysis step 121 may in particular comprise, without limiting the invention, at least one filtration of the electrocardiogram signal ECG, for example over a bandwidth of the order of 5-60 Hz, following which a step is implemented for detecting the positions of various peaks R which are reproduced successively during the signal acquisition period, these peaks R being representative of defined cardiac characteristics.

The position of these peaks, in this case the peaks R, is considered in combination with a filtered accelerometric signal SCG obtained in a second signal analysis step 122.

The control unit is configured to carried out, in parallel with the first signal analysis step 121, the second signal analysis step 122 during which the acquired accelerometric signal ACC is analyzed.

The second signal analysis step 122 comprises at least one step of filtering the accelerometric signal ACC. More specifically, the accelerometric signal may be filtered over a bandwidth of between 5 and 100 Hz. In particular, the frequency may be of the order of 20 Hz to avoid low-frequency noise and frequencies linked to cardiac movement.

The control unit is configured to carried out a step of cross-analyzing the electrocardiogram signal, and in particular the position of the peaks R identified in the filtered electrocardiogram signal ECG_1 mentioned above, with the filtered accelerometric signal SGC resulting from the second signal analysis step 122.

The cross-analysis step consists, in a synchronization operation, in defining a time cycle origin on the basis of the appearance of a peak R on the filtered electrocardiogram signal ECG_1, and it allows the step 106 of splitting the accelerometric signal, here filtered SGC, into a plurality of time cycles 107 starting respectively at each identified peak R and having the same duration.

It may be envisaged that a fixed duration, defined in advance, is applied to each time cycle from the appearance of a peak R. In the example of embodiment of the invention described here, each time cycle beginning with the appearance of a peak R ends with the appearance of the next peak R, which thus serves to define the end of time cycle n and the beginning of time cycle n+1. In order to ensure that each time cycle has the same duration, the control unit is configured to adjust the duration of the time cycles by adding time to or subtracting time from the average duration of all the time cycles thus identified by cross-analysis of the accelerometric signal and the position of the peaks R on the electrocardiogram signal.

Such a splitting of an accelerometric signal may be seen in FIG. 3, which shows the splitting of an accelerometric signal into six time cycles 107 of the same duration.

Once the filtered accelerometric signal SCG has been segmented into time cycles 107, the identification step 108 may comprise an envelope calculation step for each time cycle 107, during which an envelope approximation 109 is made to the plot of a curve representing the cardiac signal over the entire time cycle, and this calculation may be either a squared envelope calculation or an absolute value envelope calculation.

An envelope 109 calculation of this kind for each time cycle may be seen in FIG. 4, which shows a squared envelope and an envelope in absolute value for each time cycle. It is understood that any method allowing a signal rectification may be implemented here without departing from the context of the invention.

The identification step 108 also comprises a step of identifying specific segments S1, S2 or S3 of the processed time cycle, by detecting the amplitude values of one or other of the previously calculated envelopes which deviate significantly from a flat trace of the cardiac signal. The control unit thus identifies the presence of a segment of a first type S1 when the amplitude of the signal, or more specifically the amplitude of one and/or other of the envelopes, is greater than a first threshold value. The control unit similarly identifies the presence of a segment of a second type S2 when the amplitude of the signal, or more particularly the amplitude of one and/or the other of the envelopes, is greater than a second threshold value, and the presence of a segment of a third type S3 when the amplitude of the signal, or more particularly the amplitude of one and/or other of the envelopes, is greater than a third threshold value.

A segment is defined over a given time range during which the amplitude of the envelope of the accelerometric signal is greater than the corresponding threshold value, it being understood that an amplitude of the envelope greater than the corresponding threshold value over too short a duration may be considered an artefact and is not assimilated as a characteristic segment.

In order to ensure that the first, second and third segments are correctly identified, the control unit performs these comparisons of the envelope amplitude values with a threshold value over two distinct parts of each time cycle 107.

More particularly, the control unit searches for signal amplitude values greater than said first threshold value over a first third T1 of the time cycle 107. In other words, the segments of the first type S1 are searched for and identified only in the first part of the cycle, in this case the first third T1. The first third T1 of the time cycle 107 means the period of time in the time cycle that elapses from the origin over a period equal to one third of the total duration of the time cycle.

FIG. 5, which will be described below and which illustrates a representative time cycle of the processed accelerometric signal, shows how the time cycle is split into three thirds T1, T2 and T3. It is important to understand that in this identification step, each time cycle is split into three thirds, and segments of different types are searched for in the corresponding third.

The control unit is also configured to search for envelope amplitude values greater than threshold values different from said first threshold value over the rest of the time cycle. In other words, the segments of the second type S2 and the segments of the third type S3 are searched for and identified only on the part of the time cycle complementary to the first part of the cycle, i.e. here the last two thirds T2, T3 of the time cycle 107.

The envelope amplitude values greater than a second threshold value are assimilated to be segments of the second type S2, and envelope amplitude values greater than a third threshold value and occurring after the amplitude values corresponding to segments of the second type S2 are assimilated to be segments of the third type S3.

It is notable in this identification step that the segments of the third type S3 are searched for following the detected segments of the second type S2, in the same part of the time cycle, namely the remaining part of the time cycle wherein the segments of the first type S1 have not been searched for.

The control unit is configured to implement a step of sorting 130 the identified segments, in particular to eliminate from these segments those that may be considered as artefacts, i.e. here as segments too distinct from what is expected for the patient, by means of a spectral energy calculation as previously presented, i.e. by considering a z-score of each segment. In other words, this sorting step 130 consists of an exclusion sub-step of the identification step 108, which consists of excluding segments previously identified as being characteristic segments of the identified cardiac sound, as soon as they are classified during this sorting step as being artefacts.

The segments identified here are considered to be artefacts if the z-score associated with them is greater than 3.

At the end of this identification step 108, the control unit is able to store all the segments of first type S1 identified over all the time cycles 107 in a first database, and all the segments of second type S2 and third type S3 identified over all the time cycles in a second database wherein each segment of second type is associated with the segment of third type present in the same time cycle.

It should be noted that alternatively, in an embodiment to be described below, the control unit may be configured to store the third type segments S3 in a third database, separately from the second type segments S2.

In this first embodiment, the step 110 of selecting the coherence of the identified segments, which is implemented after the identification step 108, is split into two sub-steps carried out in parallel, during each of which all the previously identified segments of the same type are processed.

A first sub-step 1101 consists of cross-correlating all the segments of the first type identified previously in order to select the coherent segments of the first type which will subsequently be used to calculate an average segment of the first type.

More particularly, the first sub-step 1101 comprises a step of cross-correlating 1101_1 each of the segments of the first type S1 to define a segment of the first reference type S1_ref. In other words, each of the segments of the first type S1 is correlated with all the other segments of the first type and an average correlation score is assigned to each time cycle on the basis of the correlation scores established as a function of the coherence of the segment of the first type S1 associated with this time cycle with each of the other segments of the first type associated with the other time cycles. The reference segment, and the corresponding reference time cycle, is the one that best cross-correlates with all the others. Then, during the first sub-step 1101, a coherence sorting step 1101_2 is carried out, by comparing each segment of first type S1 present in the database, and not eliminated by the previous spectral energy analysis, with the reference segment of first type S1_ref so as to define a correlation index. The control unit is configured so as not to retain the segments of the first type for which the correlation index is less than a defined threshold, which may be of the order of at least 50% and here of the order of 60%, without this being restrictive of the invention.

Finally, in a resetting step 1101_3 of this first sub-step 1101, the control unit is configured to reset in time, on the reference first type segment S1_ref, each of the first type segments S1 which have been retained, i.e. each of the segments which have not been excluded from the analysis by the spectral energy calculation or by the coherence sorting step. The resetting is carried out in such a way as to obtain a maximum correlation between the reset segments and the corresponding reference segment.

On the basis of these reset segments and the reference segment, the method then implements the step of calculating 112 an average segment, in this case the step of calculating 1121 an average segment of first type S1_moy, representative of each of the identified and non-excluded segments of first type S1. This calculation step consists of carrying out, for each instant in the time cycle, for example each millisecond, an averaging of the amplitude values of each of the segments of the first type S1.

The control unit is configured to store the average first type segment S1_moy associated with the analysis of the accelerometric signal.

Simultaneously with carrying out the first sub-step 1101, the control unit carries out a second sub-step 1102. This second sub-step 1102 comprises the same steps as the first sub-step 1101, namely a cross-correlation calculation step 1102_1 to define a segment of second reference type S2_ref, a coherence sorting step 1102_2 with respect to the reference segment, a time resetting step 1102_3 and is also followed by a step 1122 of calculating an average segment. Once again, this second sub-step, and the various steps it comprises, is only performed on the segments that have passed the spectral energy analysis step, which means that the calculations may be concentrated on the useful segments only.

In this first embodiment, the second sub-step 1102 is carried out by considering only the segments of the second type S2, the segments of the third type S3 being retained or discarded depending on what is done for the segment of the second type S2 present in their corresponding time cycle.

More specifically, segments, and therefore time cycles, are sorted by spectral energy by calculating the z-score of the segments of the second type S2. If a segment of the second type has a z-score below a threshold value, here equal to 0.3, the entire time cycle, i.e. the segment of the second type and the associated segment of the third type, is excluded.

In a similar way, the cross-correlation calculation step 1102_1 is performed on the basis of the segments of the second type, so that a reference segment of the second type S2_ref is chosen. Each of the second type segments S2 stored in the second database, and not excluded by the spectral analysis, is then compared with the reference second type segment S2_ref. In accordance with what has been described for the first sub-step 1101, the control unit removes from the analysis those segments of the second type for which the comparison with the reference segment of the second type results in a correlation threshold deemed insufficient, which by way of example here is of the order of 60% without this value being limiting. However, here, this exclusion of the segments of the second type S2 also has the effect of excluding the segments of the third type S3 identified in a time cycle 107 corresponding to an excluded segment of the second type S2.

The segment average value calculation step is then performed for each of the segment types, i.e. the second type S2 segments and the third type S3 segments.

The control unit is configured to store the average second type segment S2_moy and the average third type segment S3_moy, associated with the analysis of the accelerometric signal. These average segments representative of the processed accelerometric signal ACC-T are then used in the step of determining a risk of cardiac decompensation 114, in particular by analyzing changes in the amplitudes of the segments from one signal to the next.

The control unit may be configured, for the purposes of displaying the signal, to reconstruct a time cycle representative of the processed accelerometric signal ACC-T, on the basis of the segment of first mean type S1_moy, the segment of second mean type S2_moy and the segment of third mean type S3_moy.

FIG. 5 illustrates by way of example an average time cycle 107, comprising a segment of a first average type S1_moy, present in the first third T1 of the time cycle, as well as a segment of a second average type S2_moy and a segment of a third type S3_moy which are present in the remainder of the time cycle, namely in the second thirds T2 and T3.

It is understood that this first embodiment is special in that it allows a signal to be reconstituted with an average third type segment S3_moy, without the cross-correlation calculations being carried out on these types of segments, but on second type segments, with larger amplitudes and therefore allowing a more reliable cross-correlation and coherence calculation.

With reference to FIG. 6, a second embodiment will now be described, wherein the second sub-step 1102 differs from the second sub-step of the first embodiment in that the steps of cross-correlation to determine a reference segment and sorting by comparison of the segments with the reference segment are carried out both for the segments of the second type and for the segments of the third type S3. The average third type segment S3_moy is calculated by the control unit on the basis of a sorting of the identified third type segments which is carried out independently of the sorting of the identified second type segments.

The steps prior to the identification step 108 are substantially the same here, so they have not been shown in FIG. 6.

As described above, the step 110 of selecting the coherence of the identified segments, which is implemented after the identification step 108, is divided into several sub-steps carried out in parallel, during each of which all the previously identified segments of the same type are processed. As before, a first sub-step 1101 consists of cross-correlating all the segments of the first type identified previously in order to select the coherent segments of the first type and subsequently calculate an average segment of the first type S1_moy.

As mentioned previously, in this second embodiment, the control unit is configured to store the third type segments S3 in a database separate from the database wherein the second type segments S2 are stored.

In this second embodiment, the segment coherence selection step 110 is divided into three sub-steps. Simultaneously with carrying out the first sub-step 1101, the control unit performs a second sub-step 1102 and a third sub-step 1103. The second sub-step 1102 and the third sub-step 1103 respectively comprise the same steps as the first sub-step 1101, namely a cross-correlation calculation step 1102_1, 1103_1 to define a reference segment S2_ref, S3_ref, a coherence sorting step 1102_2, 1103_2 with respect to the reference segment and a time resetting step 1102_3, 1103_3 and each of these two sub-steps is respectively followed by a step 1122, 1123 of calculating an average segment S2_moy, S3_moy.

It is thus understood that in this second embodiment, unlike the first embodiment, a reference third-type segment S3_ref is determined by a cross-correlation calculation specific to the third-type segments S3. The second sub-step 1102 is carried out considering only the segments of the second type S2 and the third sub-step 1103 is carried out considering only the segments of the third type S3, the segments being retained or excluded in each sub-step solely as a function of what is done in the corresponding sub-step.

By way of example, in this second embodiment, it is possible for the segment of third type S3 present in a defined time cycle 107 to be retained because it is coherent with the reference segment of third type S3_ref while simultaneously the segment of second type S2 present in this same defined time cycle 107 is excluded because it is not coherent with the reference segment of second type S2_ref. The averaging step 1122 at the end of the second sub-step 1102 may thus be based on a number of segments of the second type S2 which is different from the number of segments of the third type S3 on which the averaging step 1123 at the end of the third sub-step 1103 is based.

Here again, a sorting 130 of the segments by spectral analysis is carried out, in this case both for the segments of the first type in the first sub-step 1101, for the segments of the second type S2 in the second sub-step 1102 and for the segments of the third type S3 in the third sub-step 1103.

This sorting serves the same purpose as in the first embodiment, namely to avoid integrating in the cross-correlation calculations segments that are clearly atypical and should be considered as artefacts. This reduces the calculation time associated with each sub-step.

In accordance with what was said for the first embodiment, the control unit is then able to store the average segments of each of the types of segments and to restore them for the step of determining a risk of cardiac decompensation 114, during which in particular the change in the amplitudes of the segments from one signal to another is analyzed.

Here again, it is possible to reconstruct, for display purposes, a processed accelerometric signal ACC-T on the basis of the average first type segment S1_moy, the average second type segment S2_moy and the average third type segment S3_moy.

It is understood that this second embodiment is special in that it allows a signal with an average third type segment S3_moy to be reconstituted, based on the direct analysis of these third type segments.

The invention as it has just been described allows to achieve its stated aim, i.e. to efficiently process an accelerometric signal acquired from a patient in order to define a processed signal allowing the reliable determination of an increase or a decrease in the amplitude of certain data within this signal, in order to provide the practitioner with tools for diagnosing any cardiac decompensation. Variations not described here may be implemented without leaving the context of the invention, as long as, in accordance with the invention, they form part of a determination method allowing in particular to define average segments of at least three types representative of an accelerometric signal acquired on the patient.

By way of a non-exhaustive example, a possible variant is to provide an additional step wherein the number of segments of each type that are retained following a step in the method is analyzed and this number is compared with a threshold value. If the number of segments of one of the types, for example the number of segments of the first type, is less than said threshold value, the acquired signal is considered to be of poor quality and is not retained in order to calculate average segments, so that this acquired signal is not processed with a view to the step of determining a risk of cardiac decompensation. The threshold value may, for example, be a number corresponding to 50% of the number of time cycles resulting from the splitting of the accelerometric signal. The method step following which this additional step is implemented may in particular be the segment identification step and the exclusion sub-step with calculation of a spectral analysis score. The numbers of segments of the first type S1, of the second type S2 and of the third type S3, which are respectively retained following this exclusion sub-step, are compared with said threshold value and a decision to continue the method, and in particular to initiate the coherence sorting step, for which a large number of cross-correlation calculations have to be performed, is taken as a function of the result of this comparison. As mentioned above, if one of the numbers of segments of a given type is less than the threshold value, the acquisition of the accelerometric signal as a whole is deemed unreliable and no calculations are subsequently performed on the basis of this acquired accelerometric signal.

Another possible variant would be to provide another exclusion sub-step, which would take place during the time cycle splitting step and which would consist of excluding from the method the time cycles whose duration, defined in particular by the interval between two successive peaks R, is much longer than the mean value, or the median value, of the durations of the other time cycles. For example, the time cycles with an original duration greater than 1.7 times said mean or median value are excluded from the rest of the method and are therefore neither reset to the mean duration of the time cycles nor analyzed to detect characteristic segments. Similarly, the time cycles with too short a duration, for example with a duration at the origin of less than 0.3 times said mean or median value, are also excluded.

1. A determination method for determining a risk of cardiac decompensation of a patient, during which a control unit performs a step of processing an accelerometric signal acquired from the patient in order to implement a step of determining a risk of cardiac decompensation based on the study of a plurality of markers including segments of a processed accelerometric signal resulting from said processing step wherein the method of processing the accelerometric signal implemented by the control unit comprises:
splitting the accelerometric signal into time cycles of a same duration;
identifying, in each time cycle, segments characteristic of the cardiac sound, with a first search for a first type of segment in a first defined part of the time cycle and a second search for at least a second type of segment and a third type of segment in a second defined part of the time cycle,
selecting a coherence of the segments identified, at least for the first types of segments and the second types of segments, said selection step consisting of cross-correlating all the segments of the same type identified beforehand in order to be able to deduce a reference segment for this type of segment and to be able, in a second step, to keep only the segments which come closest to the reference segment, which are the coherent segments of each of the types of segment, and
calculating an average segment of a first type, an average segment of a second type and an average segment of a third type, the average calculation being based on all the segments of a first type selected, respectively all the segments of a second type selected, respectively all the segments of a third type selected,
wherein the determining step comprises at least one step of determining an increase in the amplitude of the first type of segment relative to data in memory and/or a decrease in the amplitude of the third type of segment relative to data in memory, the control unit being configured to store the average segment of the first type associated with the analysis of the accelerometric signal, and on the other hand, to store the average segment of the second type and the average segment of the third type, associated with the analysis of the accelerometric signal, these average segments representative of the processed accelerometric signal being used for the determining step, during which the changes in the amplitudes of the segments from one signal to the next are analyzed.

2. The determination method according to claim 1, wherein the identifying step comprises a sub-step of excluding segments previously identified as being segments of a first, second, or third type, and classified during the exclusion sub-step as being artefacts.

3. The determination method according to claim 2, wherein the exclusion sub-step comprises a step of calculating a spectral analysis score, the segments presenting a score greater than a threshold value being identified as being artefacts and excluded.

4. The determination method according to claim 1, wherein the processing step performed for each of the signals detected on one of the three acquisition axes of the accelerometric signal or else for an overall signal resulting from the combination of the signals acquired on each of the axes.

5. The determination method as claimed in claim 1, further comprising processing of an electrocardiogram signal acquired from the patient.

6. The determination method according to claim 5, wherein the step of splitting the signal comprises an operation of synchronizing the accelerometric signal on the basis of the electrocardiogram signal (ECG).

7. The determination method according to claim 1, wherein the identifying step further comprises calculating an envelope of the accelerometric signal, a segment being identified when the amplitude of the envelope is greater than a determined threshold in a defined part of the time cycle.

8. The determination method according to claim 1, wherein the first defined part of the time cycle in the identifying step is equal to the first third of the duration of said time cycle, the second defined part of the time cycle being equal to the last two thirds of said time cycle (107).

9. The determination method according to claim 1, wherein the selecting step further comprises at least two series of correlation calculations performed in parallel, including a first series of correlation calculations performed on all the segments of the first type , and a second series of correlation calculations performed on all the segments of the second type.

10. The determination method according to claim 9, wherein each series of correlation calculations performed for a given type of segment comprises a first calculation of the cross-correlation between all the segments identified as being segments of this type, in order to define a reference segment for the type of segment, followed by calculating the correlation of each of the segments identified as being segments of the type with respect to the reference segment.

11. The determination method according to claim 10, wherein the segments selected in each series of correlation calculations are the segments which have a correlation index with the reference segment which is greater than or equal to a predetermined correlation threshold.

12. The determination method according to claim 9, wherein the selection step comprises only two series of correlation calculations performed in parallel, including a first series of correlation calculations performed on all the segments of the first type and a second series of correlation calculations performed on all the segments of the second type.

13. The determination method according to claim 9, wherein the selection step comprises three series of correlation calculations performed in parallel, including a first series of correlation calculations performed on all the segments of the first type, a second series of correlation calculations performed on all the segments of the second type, and a third series of correlation calculations performed on all the segments of the third type.

14. The determination method according to claim 10, wherein, during the selecting step and before the step of calculating the average segments, a step of time resetting the selected segments with the corresponding reference segment is performed for each type of segment.
